(19) United States
(12) Patent Application Publication
(10) Pub. No.: US 2019/0374786 A1
(43) Pub. Date: Dec. 12, 2019

(54) LOW-POWERED ELECTROMAGNETIC BRAIN STIMULATION DREAMING APPARATUS AND METHOD

(71) Applicant: Mitchell Dart Jorgensen, American Fork, UT (US)

(72) Inventor: Mitchell Dart Jorgensen, American Fork, UT (US)

(21) Appl. No.: 16/435,459

(22) Filed: Jun. 7, 2019

Related U.S. Application Data
(60) Provisional application No. 62/682,143, filed on Jun. 7, 2018.

(51) Int. Cl. A61N 2/00; A61N 2/02; A61M 21/02

(52) U.S. Cl. CPC A61N 2/006; A61M 2021/0055; A61M 21/02; A61N 2/02

(57) ABSTRACT

A brain stimulation apparatus and method making use of electromagnetics in the form of solenoids, in some embodiments, to stimulate brain function during sleep using oscillating low-powered electromagnetic fields to activate select regions of the brain of a subject which interact weakly with said regions, including, in some embodiments, the prefrontal cortex, the hippocampus, visual cortex, and posterior cortical hot zone.

### BACKGROUND OF THE INVENTION

### Field of the Invention

This invention relates to brain stimulation and dreaming and more particularly relates to a device which uses electromagnetic fields to stimulate brain activity during sleep.

### Description of the Related Art

Dreams typically occur in the rapid-eye movement (REM) stage of sleep when brain activity is high and resembles that of being awake. REM sleep is revealed by, continuous movements of the eyes during sleep. It is commonly accepted that the intensity of dreams during REM sleep can be increased or decreased by the dopaminergic cells of the ventral tegmental area. Drugs which block dopaminergic activity, such as haloperidol, inhibit unusually frequent, and vivid dreaming, while increase of dopamine stimulates excessive vivid dreaming and nightmares.

Many individuals wish to control the intensity of their dreams or exercise some control over them, but without the use of drugs. It is not uncommon for individuals suffering from nightmares, or even the lack of dreams, to seek help from specialists in managing dreaming—sometimes in connection with other sleep-related health conditions, such as sleep apnea.

Recent studies suggest that lucid dreaming (awareness of dreams while dreaming) might be associated with higher than average brain activity over frontal regions of the brain during rapid eye movement (REM) sleep. Other areas of the brain are also known to influence sleep behavior including the hippocampus and posterior cortical hot zone.

It is possible that these areas of the brain can be manipulated, activated, stimulated, or deactivated by inducing electric current in these regions using electromagnetic fields. The activity of the dorsolateral prefrontal cortex (DLPFC) is thought during REM sleep to increase when exposed to electrical currents.

Although devices and methods exist in the art which use non-invasive methods to stimulate, activate, and deactivate activity in the brain during sleep, these devices suffer from many inefficiencies and disadvantages, including that they almost exclusively make use of electrodes which must make direct contact with the scalp or skin and function by sending electrical current into cranium rather than issuing electromagnetic fields from a solenoid. Patients must often shave their heads so that their skin can make contact with the electrodes.

Especially in use cases regarding sleeping and dreaming, users often report feeling disturbances or negative electrical sensations due to the use of electrodes. Dream stimulation researchers have been unable to collect and conclude accurate research data due to a number of volunteers in dream studies sensing the electrical sensations and waking up. Typical devices in the art use high intensity voltage and/or wattage, which cause electrical or electromechanical components in the device to heat up. This heating of the electrodes and other hardware is a problem that creates discomfort for subjects, potentially endangers subjects, and limits the duration of stimulation at high intensities and frequencies. Questions regarding user safety and potential damage between the electrodes and the contact tissues plague the current art.

Additionally, use of medium- or high-powered electromagnetic fields typically causes a subject to wake up.

There exists a need in the art for an apparatus and method which may be used to impart electromagnetic fields across the brain to further study the effects of dreaming, but which does not require the use of electrodes or anodes which engage the skin directly. A device which is not worn by the subject may be of use in the industry. There exists a further need in the art for an apparatus which makes use of low-powered oscillating magnetic signals that pose limited risk to tissues, DNA, and cells of the user.

The present invention seeks to remedy these deficiencies in the art.

### SUMMARY OF THE INVENTION

From the foregoing discussion, it should be apparent that a need exists for a cranial brain stimulations apparatus. Beneficially, such an apparatus would overcome many of the difficulties with prior art by providing a single apparatus which does not use electrodes or anodes to engage a subject's skin or cranium.

The present invention has been developed in response to the present state of the art, and in particular, in response to the safety problems and needs in the art that have not yet been fully solved by currently available aparati. Accordingly, the present invention has been developed to provide a method of inducing lucid dreaming, the steps of the method comprising: positioning one or more solenoids within six feet of the cranium of a subject during sleep, the solenoids operable to induce a magnetic field of sufficient magnetic flux density (B) and magnetic field strength (H) to be measurable at the cranium by a magnetometer; and oscillating power to the solenoids to vary peak values of electric field state envelopes of the magnetic flux density using pulse width periods of less than 0.25 seconds.

The magnetic field may fluctuate between 0.1 and 6 picotesla (pT). The magnetic field may alternatively fluctuates between 0.00005 milliGaus (mG) and 10 milliTesla (mT).

When a power signal to the solenoids is graphed, the oscillating power may create one of: a bidirectional waveform, a bidirectional Sine-waveform, a phase-modulated waveform, and a clock pulse unidirectional waveform. The bidirectional Sine-waveform may indicate a magnetic flux density of less than 6 milliTesla (mT). The magnetic field may be operable to stimulate a brain of the subject.

In some embodiments, the magnetic field may fluctuate between 1 mG and 7.4 milliTesla (mT).

The method may further comprise continuously oscillating power to the solenoids for an extended dream stimulatory period, the dream stimulatory period defined by one of: an instruction in computer readable memory forming part of a dream stimulatory program executed by a processor; and in response to sensory feedback data falling below a predetermined threshold, the sensory feedback data derived from one or more of the following sensors: an EEG sensor; an infrared optical pulse measurement sensor; a heart rate-monitor; a 3D accelerometer; and and a thermometer.

The peak values of electric field state envelopes of the magnetic flux density may fluctuate from one electronic field state envelope to another such that peak values of electric field state envelopes of the magnetic flux density are non-uniform between all electric field state envelopes sequentially applied during the dream stimulatory period.

The method may further comprise deriving sensory feedback data from one or more of the following sensors: an EEG sensor; an infrared optical pulse measurement sensor; a heart rate-monitor; a 3D accelerometer; and and a thermometer.

The one or more solenoids may position within a housing of a substantially ovoid head apparatus adapted to circumscribe the cranium of the subject, the annular head apparatus comprising a heat-resistant material separating the solenoids from direct engagement with the subject's skin.

In some embodiments, the one or more solenoids position at a point on the substantially ovoid head apparatus in closest proximity to one of the following regions of the brain: dorsolateral prefrontal cortex, the visual cortex, hippocampus and posterior cortical hot zone.

The method may further comprise: selectively activating the one or more of the solenoids in closest proximity to one of the following regions of the brain: dorsolateral prefrontal cortex, the visual cortex, hippocampus and posterior cortical hot zone; wherein said selective activation is in response to one of: an instruction in computer readable memory forming part of a dream stimulatory program executed by a processor; and sensory input derived from one or more of the following sensors: EEG sensor, infrared optical pulse measurement sensor; a heart rate-monitor; a 3D accelerometer; and and a thermometer.

A system for inducing lucid dreaming in a subject is also provided, the system comprising: persistent computer-readable memory; a control module comprising a processor, the control module configured to execute a dream stimulatory program stored in computer-readable instructions in the persistent computer-readable memory; an oscillator module configured to oscillate power to one or more solenoids positioned within six feet of the cranium of a subject during sleep, the solenoids operable to induce a magnetic field of sufficient magnetic flux density (B) and magnetic field strength (H) to be measurable at the cranium by a magnetometer; wherein the oscillator module is further configured to vary peak values of electric field state envelopes of the magnetic flux density using pulse width periods of less than 0.25 seconds, wherein the magnetic field fluctuates between 0.00005 mG and 10 milliTesla (mT).

When a power signal to the solenoids is graphed, the oscillating power may create one of a bidirectional waveform, a bidirectional Sine-waveform, a phase-modulated waveform, and a clock pulse unidirectional waveform.

The bidirectional Sine-waveform may indicate a magnetic flux density of less than 6 milliTesla (mT). The system may further comprise: a positioner module configured to position one more solenoids in closest proximity to one of the following regions of the brain: dorsolateral prefrontal cortex, the visual cortex, hippocampus and posterior cortical hot zone.

The transmitter may further be configured to transmit sensory input from the plurality of sensors wirelessly using using Bluetooth® technology.

A second method of inducing lucid dreaming is provided, the steps of the method comprising: positioning one or more solenoids within six feet of the cranium of a subject during sleep, the solenoids operable to induce a magnetic field of sufficient magnetic flux density (B) and magnetic field strength (H) to be measurable by a magnetometer; and oscillating power to the solenoids to vary peak values of electric field state envelopes of the magnetic flux density using pulse width periods of less than 0.25 seconds; wherein the magnetic field fluctuates between 0.00005 milliGauss (mG) and 10 milliTesla (mT).

When a power signal to the solenoids is graphed, the oscillating power creates one of a bidirectional waveform, a bidirectional Sine-waveform, a phase-modulated waveform, and a clock pulse unidirectional waveform. The bidirectional Sine-waveform may indicate a magnetic flux density of less than 6 milliTesla (mT).

The magnetic field may be operable to stimulate one or more of the follow regions of a brain of the subject: dorsolateral prefrontal cortex, the visual cortex, hippocampus and posterior cortical hot zone.

These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only some embodiments of the invention and are not therefore to be considered to be limiting of its scope, the invention will be described and explained with additional specificity and detail through the use of the accompanying drawings, in which:

FIG. 1 is an environmental upper perspective view of a system of stimulating brain function during sleep in accordance with the present invention;

FIG. 2A is an upper perspective view of an array of electromagnets of a cranial brain stimulation apparatus in accordance with the present invention;

FIG. 2B is an upper, forward perspective view of a cranial brain stimulation apparatus in accordance with the present invention;

FIG. 3 is an upper, forward perspective view of a cranial brain stimulation apparatus in accordance with the present invention;

FIG. 4 is a side perspective view of an electromagnet in accordance with the prior art;

FIG. 5A is a forward perspective view of a cranial brain stimulation apparatus in accordance with the present invention;

FIG. 5B is an isometric perspective view of a cranial brain stimulation apparatus in accordance with the present invention;

FIG. 6 is an upper, forward perspective view of a disassembled cranial brain stimulation apparatus in accordance with the present invention;

FIG. 7 is a flow chart of a method stimulating a brain during sleep through the cranium in accordance with the present invention;

FIG. 8 is a block diagram of a system for stimulating a brain during sleep through the cranium in accordance with the present invention; and

FIG. 9 illustrates the regions of the subject's brain potentially stimulated in accordance with the present invention.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided to convey a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

It is an object of the present invention to provide an apparatus making use of low-powered oscillating magnetic signals that pose limited risk to tissues, DNA, and cells of the user, and which makes use of electromagnetic fields instead of direct current to stimulate brain function during sleep.

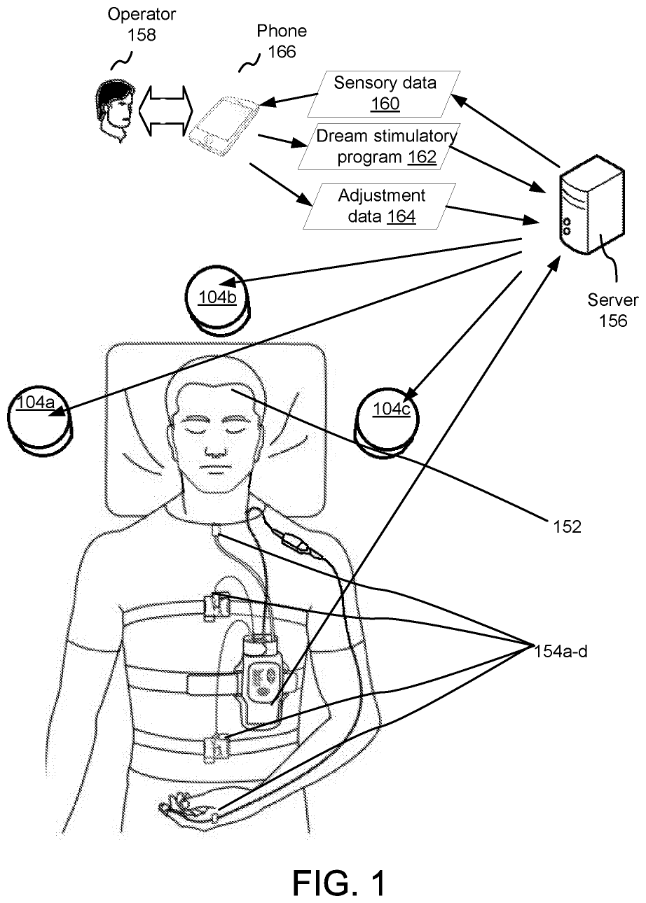

FIG. 1 is an environmental upper perspective view of a system 100 of stimulating brain function during sleep in accordance with the present invention.

The system 100 generates weak fluctuating magnetic fields around the cranium of a subject 152 (i.e., “person” or “patient”). These fields are generating using one or more solenoids 104; which, in the shown embodiment, are placed in proximity to the subject's 152 cranium. In various embodiments, the solenoids 104 may be placed in lateral proximity to the subject 152 on a table, floor, stand or the like. The solenoids 104 may be placed within 6 to 10 feet of the subject, usually clear of any intervening structures which would disrupt the magnetic field and/or changed magnetic field strength.

Various form of solenoids 104 and/or electromagnets may be used, some housed within a cylindrical metallic or polymeric housing.

The system 100 includes the various hardware methods, and software computer program products, necessary to impart repeating regular or irregular supplies of power to the solenoids 104 with the objection of using signals in the form of electromagnetic fields to stimulate the brain of the subject 152 during sleep, through use of the toroidal-shaped inductor(s) or wire(s) within the solenoids 104a-c.

The solenoids 104 can be powered and controlled in both wired and wireless configurations. In both configurations, the solenoids 104 may be in logical communication with a data processing device (DPD), such as a server 156.

The solenoids 104 can be activated in unison or independently of one another in accordance with a predetermined dream stimulatory program in computer-readable memory within the DPD 156. The dream stimulatory program 162 may comprise a variety of power settings which are executed sequentially (or recursively) to produce a signal in form of an oscillating or varying electromagnetic field around the cranium of the subject 156, which signal is used to effectuate stimulation upon the dreaming region of the subject's 156 brain.

The DPD 156 may comprise any system, apparatus, or computer program running on one or more data processing devices (DPDs), such as a server, computer workstation, router, mainframe computer, or the like. In various embodiments, the DPD comprises one or more processors. The processor is a computing device well-known to those in the art and may include an application-specific integrated circuit (“ASIC”) within a physical head apparatus circumscribing the subject's 152 head or cranium.

The solenoids 104 may be in logical communication with the DPD 156 over a WAN (wide area network) or LAN (local area network).

The solenoids 104 may cycle through different power settings inputs. The pulse width frequencies may be 4 Hz or more.

These oscillating signals from the solenoids effect REM sleep stages in the subject 152. The frequencies of, and wave form of, the signal patterns can include a bidirectional waveform, a bidirectional Sine-waveform, a phase-modulated waveform, and a clock pulse unidirectional waveform.

The subject 154a-d is fitted during sleep with a plurality of sensors 154a-d further described below. The sensors 154a-d communicate sensory data 160 to the DPD 156 which may be relayed in real time to an operator 158 who may be monitored the subject 152 during sleep cycles.

The sensors 154 can include EEG sensors, infrared optical pulse measurement devices, heart rate-monitors, 3D accelerometers, gyroscope, body temperature sensors, and other methods of biological feedback. All sensors and feedback may be connected and integrated for use with the mobile app software, desktop software program and the online user interface.

The signal imparted to the subject 152 can be produced, modified, terminated, restarted or intensified by an operator 158 in some embodiments. The operator 158 may have control over the dream stimulatory program being executed by the DPD 156.

In some embodiments, the subject 152 creates the dream stimulatory program 162 before falling asleep and puts the dream stimulatory program 162 into computer-readable memory before sleep. In other embodiments, the DPD automatically adjusts the dream stimulatory program 162 during sleep in response to sensory data 160 derived in real time from the subject 152 during sleep. In still further embodiments, the dream stimulatory program 162 is changed manually during sleep by the operator 158 who sends adjustment data 164 to the DPD 156 from a DPD 166.

The dream stimulatory program 162 may be configured, or written, by the operator 158 on a table computer 166 or other DPD under the control of the operator 158. The operator 158 may be the subject 152 in some embodiments, who writes their own dream stimulatory program.

In various embodiments, the DPD 156 is a computer, phone, or controller used to control the solenoids 104 and execute the dream stimulatory program 162. The dream stimulatory program 162 may be configured using a mobile application, computer software, API, or online interface.

Virtual buttons and controls may be displayed on the touch display in close proximity to the subject 152 for facilitating interaction with the wearer or user. In various embodiments, the subject 152 is prompted to pick from one of a plurality of virtual buttons. In other embodiments, other types of virtual controls are displayed on the touch display including dials and sensory data 160.

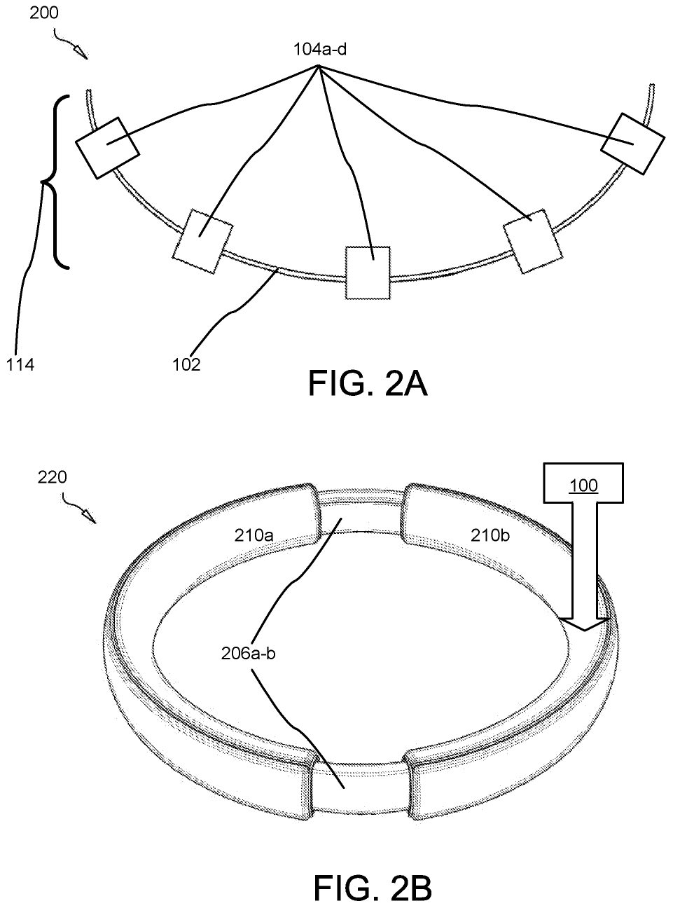

FIG. 2A is an upper perspective view of an array of electromagnets of a cranial brain stimulation apparatus 100 in accordance with the present invention.

The apparatus 200 can be constructed with one or more solenoids 104. As described above, the solenoids 104 can be placed on, nearby, or in proximity with specific regions of the subject's 152 brain.

In the shown embodiment, the solenoids 104 position on a band disposed around the head of the subject 152. The apparatus 200, or band, can be constructed in various form embodiments as either a wearable apparatus or non-wearable apparatus 200. In still further embodiments, the apparatus 200 is incorporated into a bed, enclosure or walls of a room itself.

FIG. 2B is an upper, forward perspective view of a cranial brain stimulation apparatus 220 in accordance with the present invention.

The brain stimulation device 220 may comprise a plurality of arcuate housings 210a-b, which may comprise be adapted to contour a subject's 152 head. The housings 210a-b define, in the shown embodiment, a hollow interior recesses in which the telescoping slides 206 travel.

The housings 210a-b may be formed from polymeric, metallic, or metal alloys, or even organic, or metallic materials, including steel, nylon and leather. The housings 210a-b may be formed as a single integrated piece, or be formed from a plurality of components, usually mold injected, which are affixed together.

The one or more solenoids 104 positioning within the housing 210 may be separated from the skin of the subject 152 by a heat-resistant material separating the solenoids 104 from direct engagement with the subject's 152 skin.

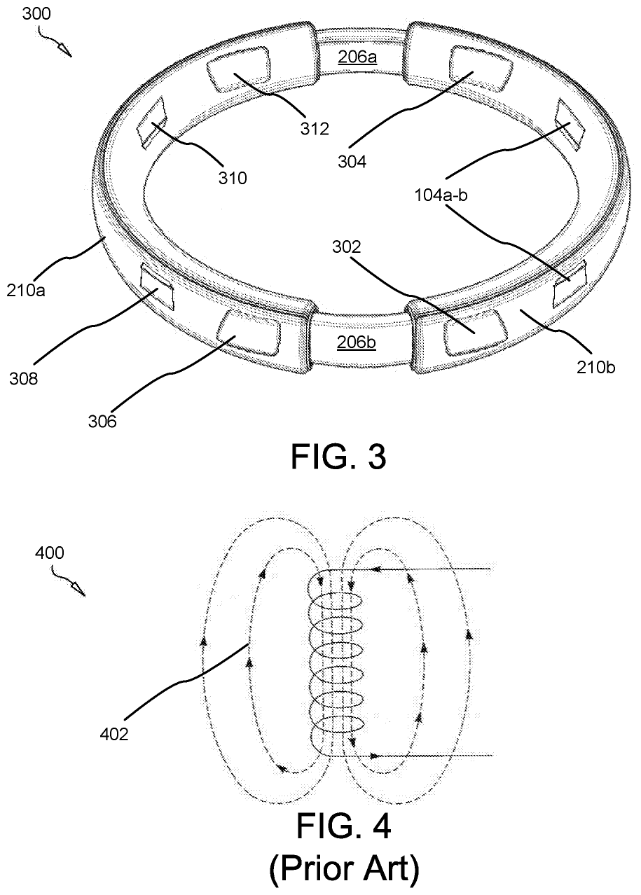

FIG. 3 is an upper, forward perspective view of a cranial brain stimulation apparatus 300 in accordance with the present invention.

Depressible buttons may position around the housings 210 which are adapted to increase or decrease the strength of magnetic fields created by the solenoids 104 and/or the frequencies at which those field oscillate, including frequency and positioning of the solenoids 104.

In various embodiments, solenoids 104 positioning over some regions of the brain may be operated at different frequencies than those solenoids 104 positioning over other regions the brain of the subject 152.

The band 220 may comprise a EEG sensor 304, a power supply 310, a transceiver 308, an infrared optical pulse measurement device 306, a heart rate-monitor 312, and a thermometer 302. When sensory date a 162 is measured indicative of sleep patterns, the apparatus 220 may be configured to automatically activate the solenoids 104 and/or execute the dream stimulatory program.

The transceiver 308 may comprises means for relaying and receiving electrical signals enabling device-to-device communication (meaning wireless transmission of the dream stimulatory program 162 and/or sensory data 160. The apparatus 220 may be configured to make use of the Bluetooth® protocols and procedures enabling device-to-device intercommunication connectivity. This functionality may be provided by incorporating the Bluetooth Intercom Profile® and/or the Bluetooth Telephony Profile®, or other wireless technologies known to those of skill in the art.

This communication may be in accordance with core specifications of one or more subsets of Bluetooth® profiles, wherein the core specifications comprise one or more of: the Cordless Telephony Profile (CTP), the Device ID Profile (DIP), the Dial-up Networking Profile (DUN), the File Transfer Profile (FTP), the Hands-Free Profile (HFP), the Human Interface Device Profile (HID), the Headset Profile (HSP), and the Intercom Profile (ICP), the Proximity Profile (PXP).

The apparatus 220 may also comprise persistent computer-readable memory. The memory may comprise a memory card, and is well-known to those of skill in the art. The memory may be insertable and removable from a positioning slot defined by the housing 210.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

The apparatus 220 may be programmable using an interface on ad DPD 166 or DPD 156.

FIG. 4 is a side perspective view of an electromagnet in accordance 400 with the prior art.

The solenoids 104 comprise electromagnets as known to those of skill in the art. An electromagnet is a type of magnet in which the magnetic field 402 is produced by an electric current. Electromagnets usually consist of wire wound into a coil. A current induced through the wire creates a magnetic field which is concentrated in the hole, denoting the center of the coil. The magnetic field 402 disappears when the current is turned off. The wire turns are often wound around a magnetic core made from a ferromagnetic or ferrimagnetic material such as iron; the magnetic core concentrates the magnetic flux and makes a more powerful magnet.

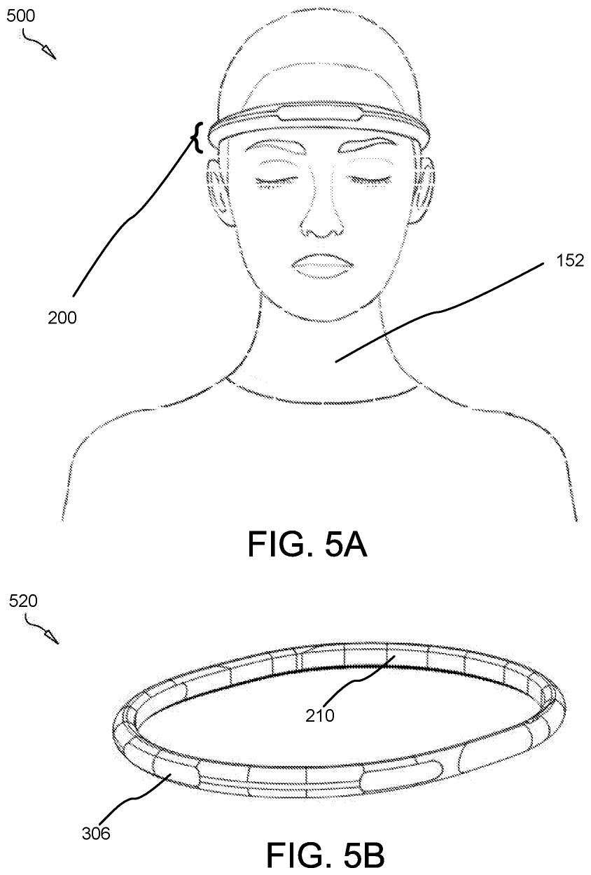

FIG. 5A is a forward perspective view of a cranial brain stimulation apparatus 500 in accordance with the present invention.

As shown, the apparatus 200 is worn by the subject 152 while asleep in some embodiments.

FIG. 5B is an isometric perspective view of a cranial brain stimulation apparatus 520 in accordance with the present invention.

In the shown embodiment, a single annular housing 210 is contoured to fit around the cranium of the user 152. The housing 210 defines a plurality of open recesses into which solenoids 104 and other electrical components are received. These recesses are covered with caps or tabs 306 which form a friction fit with the housing 210.

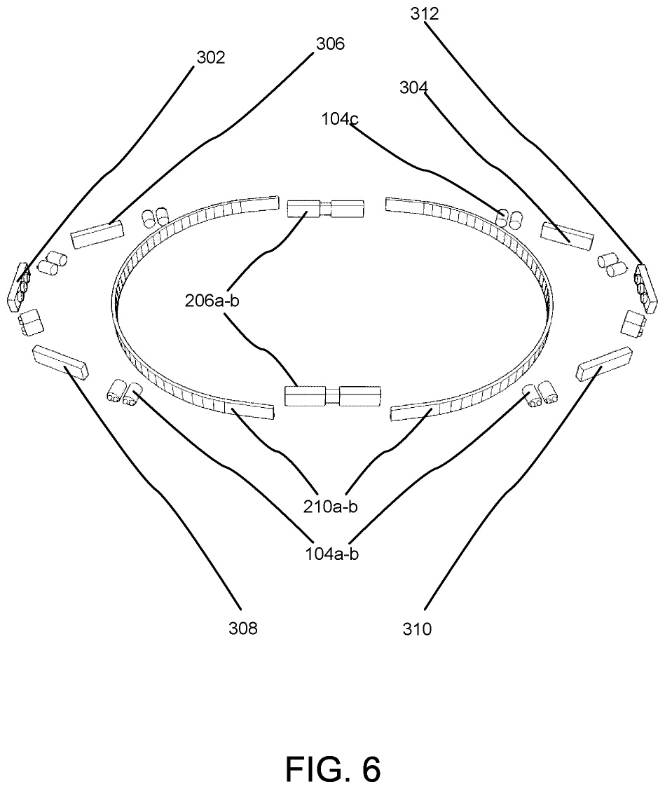

FIG. 6 is an upper, forward perspective view of a disassembled cranial brain stimulation apparatus 600 in accordance with the present invention.

The inner electrical and electromechanical components are shown, including the EEG sensor 304, the power supply 310, the transceiver 308, an infrared optical pulse measurement device 306, a heart rate-monitor 312, and a thermometer 302. When sensory date a 162 is measured indicative of sleep patterns, the apparatus 220 may be configured to automatically activate the solenoids 104 and/or execute the dream stimulatory program.

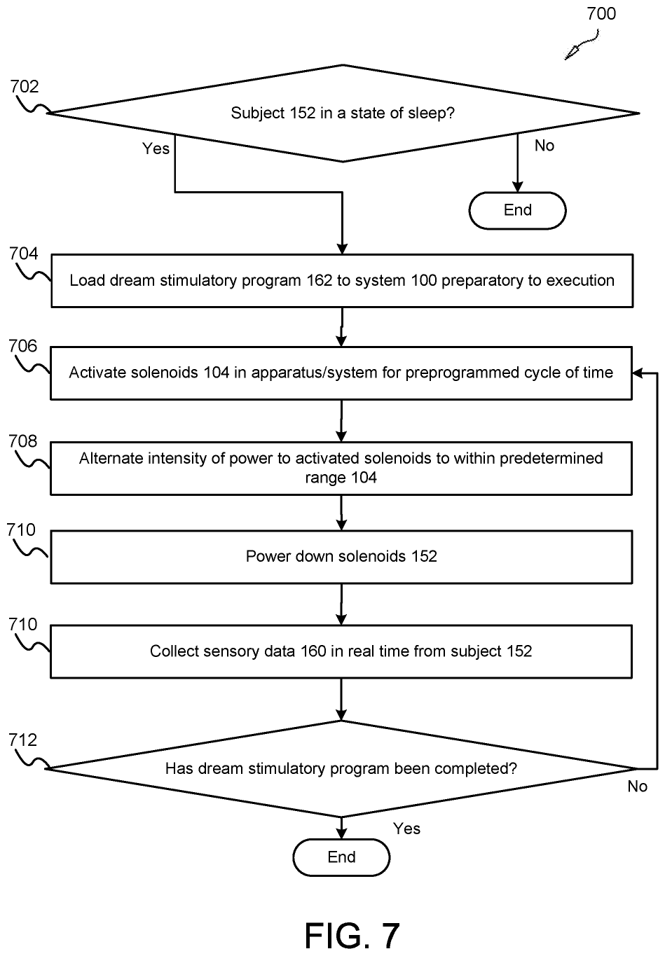

FIG. 7 is a flow chart of a method 700 of executing a dream stimulatory program in accordance with the present invention.

The method 700 begins 702 when a determination is made using sensory data 160 whether the subject 152 is in a subconscious dreaming state. If the subject 152 is in a subconscious dreaming state, the method 700 proceeds 704 where the dream stimulatory program 162 is loaded either to the apparatus 200 or to computer readable memory, such as RAM, within the system 100 prior to execution.

The solenoids 104 are activated 706 in accordance with the dream stimulatory program 162. The solenoids 104 are powered up to an intensity, in some embodiments, which is sufficient to induce a magnetic field of sufficient magnetic flux density (B) and magnetic field strength (H) to be measurable at the cranium by a magnetometer.

The magnetic field produced by each solenoid 104 402 may fluctuates between 0.1 and 6 picotesla (pT). Alternatively, the magnetic field 402 may fluctuate between 0.00005 milliGaus (mG) and 10 milliTesla (mT).

The power to the solenoids 104 is configured to vary peak values of electric field state envelopes of the magnetic flux density using periods of less than 0.25 seconds in some embodiments. The cycles may vary in frequencies from 0.05 Hz to 20 Hz in various embodiments.

The solenoids 104 may be powered down 708 completed or partially and sensory data 160 collected 710 and relayed to the DPD 166 for analysis.

If the subject 152 is determined to still be in a dreaming state and the the dream stimulatory program 162 remains incomplete, the method 700 continues at 706 in various embodiments.

The strength of the magnetic field 402 may be indicated as a bidirectional Sine-waveform, which may indicate a magnetic flux density of less than 6 milliTesla (mT). The magnetic field 402 may alternatively fluctuate between 1 mG and 7.4 milliTesla (mT).

In response to sensory data 160 indicating that sensor readings have fallen below or above a predetermined threshold for any of the following sensors, the dream stimulatory program may terminate: the EEG sensor; an infrared optical pulse measurement sensor; a heart rate-monitor; a 3D accelerometer; and and a thermometer.

In accordance with the method 700, the peak values of electric field state envelopes of the magnetic flux density may fluctuate from one electronic field state envelope to another such that peak values of electric field state envelopes of the magnetic flux density are non-uniform between all electric field state envelopes sequentially applied during the dream stimulatory period.

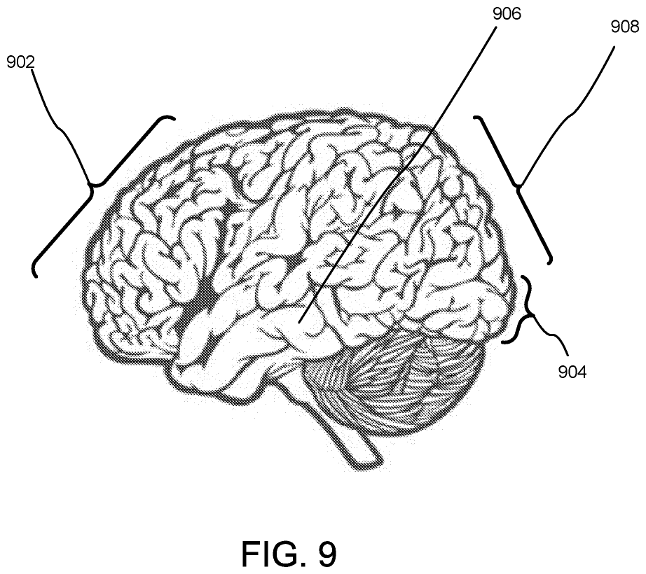

In various embodiments, one or more solenoids 104 position at a point on the substantially ovoid head apparatus 200 in closest proximity to one of the following regions of the brain: dorsolateral prefrontal cortex, the visual cortex, hippocampus and posterior cortical hot zone, which are further described below in relation to FIG. 9.

The solenoids 104 in closest proximity to select region of the brain may be the only solenoids activated in accordance with some embodiments of the present invention. As such, the method 700 may proceed selectively activating the one or more of the solenoids in closest proximity to one of the following regions of the brain: dorsolateral prefrontal cortex, the visual cortex, hippocampus and posterior cortical hot zone, wherein said selective activation is in response to one of: an instruction in computer readable memory forming part of the dream stimulatory program 162 executed by a DPD 156, 166; and sensory data 160 derived from one or more of the following sensors shown above in relation to FIGS. 1-6 and 8: EEG sensor 304, infrared optical pulse measurement sensor 306; a heart rate-monitor 312; a 3D accelerometer; and and a thermometer 302.

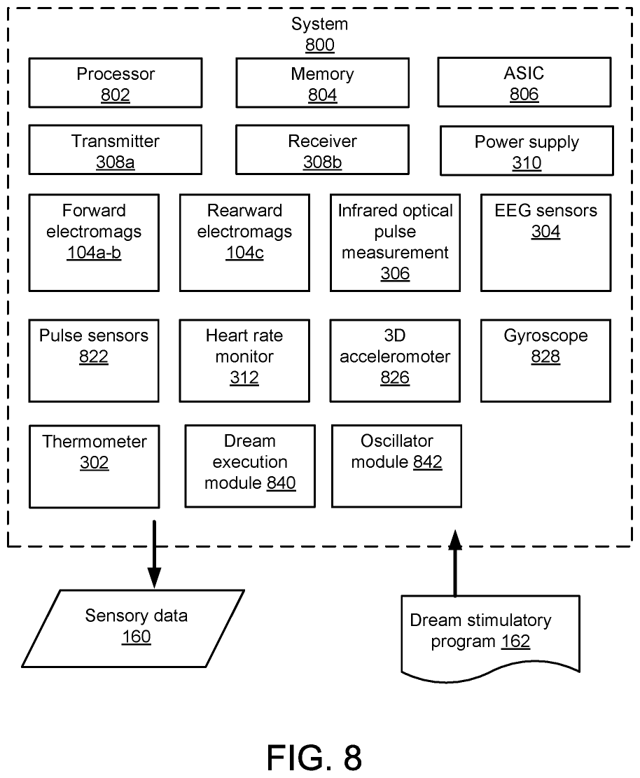

FIG. 8 is a block diagram of a system 800 for stimulating a brain during sleep through the cranium 800 in accordance with the present invention.

Each of the shown modules and components may be housing within a housing 210 or spread across a distributed system.

The dream execution module 840 (i.e., a “control module”) may be configured to execute the dream stimulatory program 162 received into computer readable memory 804.

The system 800 may comprise a processor 802 and an oscillator module 842 configured to oscillate power to one or more solenoids 104 positioned within six feet of proximity of the cranium of a subject 152 during sleep, the solenoids 104 operable to induce a magnetic field 402 of sufficient magnetic flux density (B) and magnetic field strength (H) to be measurable at the cranium of the subject 152 by a magnetometer. In other embodiments, the magnetic field 402 is measured at the solenoids 104.

The oscillator module 842 may be further configured to vary peak values of electric field state envelopes of the magnetic flux density using periods of less than 0.25 seconds, wherein the magnetic field fluctuates between 0.00005 mG and 10 milliTesla (mT).

FIG. 9 illustrates the regions of the subject's brain potentially stimulated in accordance with the present invention.

Particular regions of the brain are known to be active during lucid dreaming can be stimulated or re-activated by the solenoid 104 during non-lucid dreaming sleep states, realizing a more lucid dreaming experience for the subject 152 in which the subject exerts some control over the dreams, which may be more memorable and intense.

Shown in FIG. 9 are: the dorsolateral prefrontal cortex 902, the visual cortex 904, hippocampus 906 and posterior cortical hot zone 908.

All features and settings of apparatus can be manually configured by the user or automatically configured to the user by the included mobile application, computer software, or online interface.

Features and settings of apparatus, and electro-magnetic stimulation session design may also be chosen, selected, and modified by the user via voice command through integrated voice technologies.

The system 800 comprises a variety of sensors as shown to monitor, receive feedback, and improve the experience of the subject 152. Sensors can include EEG sensors 304, infrared optical pulse measurement devices 306, heart rate-monitors 312, 3D accelerometers, gyroscope, body temperature sensors, and other methods of biological feedback.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of inducing lucid dreaming, the steps of the method comprising:
positioning one or more solenoids within six feet of the cranium of a subject during sleep, the solenoids operable to induce a magnetic field of sufficient magnetic flux density (B) and magnetic field strength (H) to be measurable at the cranium by a magnetometer;
wherein the magnetic field fluctuates between 0.00005 milliGaus (mG) and 10 milliTesla (mT); and
oscillating power to the solenoids to vary peak values of electric field state envelopes of the magnetic flux density using pulse width periods of less than 0.25 seconds.

2. The method of claim 1, wherein, when a power signal to the solenoids is graphed, the oscillating power creates one of: a bidirectional waveform, a bidirectional Sine-waveform, a phase-modulated waveform, and a clock pulse unidirectional waveform.

3. The method of claim 2, wherein the bidirectional Sine-waveform indicates a magnetic flux density of less than 6 milliTesla (mT).

4. The method of claim 1, wherein the magnetic field fluctuates between 1 mG and 7.4 milliTesla (mT).

5. The method of claim 1, further comprising continuously oscillating power to the solenoids for an extended dream stimulatory period, the dream stimulatory period defined by one of:
an instruction in computer readable memory forming part of a dream stimulatory program executed by a processor; and
in response to sensory feedback data falling below a predetermined threshold, the sensory feedback data derived from one or more of the following sensors:
an EEG sensor;
an infrared optical pulse measurement sensor;
a heart rate-monitor;
a 3D accelerometer; and
and a thermometer.

6. The method of claim 1, wherein the peak values of electric field state envelopes of the magnetic flux density are fluctuated from one electronic field state envelope to another such that peak values of electric field state envelopes of the magnetic flux density are non-uniform between all electric field state envelopes sequentially applied during the dream stimulatory period.

7. The method of claim 1, further comprising deriving sensory feedback data from one or more of the following sensors:
an EEG sensor;
an infrared optical pulse measurement sensor;
a heart rate-monitor;
a 3D accelerometer; and
and a thermometer.

8. The method of claim 7, wherein the one or more solenoids position within a housing of a substantially ovoid head apparatus adapted to circumscribe the cranium of the subject, the annular head apparatus comprising a heat-resistant material separating the solenoids from direct engagement with the subject's skin.

9. The method of claim 8, wherein one or more solenoids position at a point on the substantially ovoid head apparatus in closest proximity to one of the following regions of the brain: dorsolateral prefrontal cortex, the visual cortex, hippocampus and posterior cortical hot zone.

10. The method of claim 9, further comprising:
selectively activating the one or more of the solenoids in closest proximity to one of the following regions of the brain: dorsolateral prefrontal cortex, the visual cortex, hippocampus and posterior conical hot zone;
wherein said selective activation is in response to one of:
an instruction in computer readable memory forming part of a dream stimulatory program executed by a processor; and
sensory input derived from one or more of the following sensors:
EEG sensor,
infrared optical pulse measurement sensor;
a heart rate-monitor;
a 3D accelerometer; and
and a thermometer.

11. A system for inducing lucid dreaming in a subject, the system comprising:
persistent computer-readable memory;
a control module comprising a processor, the control module configured to execute a dream stimulatory program stored in computer-readable instructions in the persistent computer-readable memory;
an oscillator module configured to oscillate power to one or more solenoids positioned within six feet of the cranium of a subject during sleep, the solenoids operable to induce a magnetic field of sufficient magnetic flux density (B) and magnetic field strength (H) to be measurable at the cranium by a magnetometer;
wherein the oscillator module is further configured to vary peak values of electric field state envelopes of the magnetic flux density using pulse width periods of less than 0.25 seconds, wherein the magnetic field fluctuates between 0.00005 mG and 10 milliTesla (mT).

12. The system of claim 11, wherein, when a power signal to the solenoids is graphed, the oscillating power creates one of a bidirectional waveform, a bidirectional Sine-waveform, a phase-modulated waveform, and a clock pulse unidirectional waveform.

13. The system of claim 11, wherein the bidirectional Sine-waveform indicates a magnetic flux density of less than 6 milliTesla (mT).

14. The system of claim 11, further comprising: a positioner module configured to position one or more solenoids in closest proximity to one of the following regions of the brain: dorsolateral prefrontal cortex, the visual cortex, hippocampus and posterior cortical hot zone.

15. The system of claim 11, wherein the transmitter is further configured to transmit sensory input from the plurality of sensors wirelessly using using Bluetooth® technology.

16. A method of inducing lucid dreaming, the steps of the method comprising:
positioning one or more solenoids within six feet of the cranium of a subject during sleep, the solenoids operable to induce a magnetic field of sufficient magnetic flux density (B) and magnetic field strength (H) to be measurable by a magnetometer; and
oscillating power to the solenoids to vary peak values of electric field state envelopes of the magnetic flux density using periods of less than 0.25 seconds;
wherein the magnetic field fluctuates between 0.00005 mG and 10 milliTesla (mT).

17. The method of claim 16, wherein, when a power signal to the solenoids is graphed, the oscillating power creates one of a bidirectional waveform, a bidirectional Sine-waveform, a phase-modulated waveform, and a clock pulse unidirectional waveform.

18. The method of claim 17, wherein the bidirectional Sine-waveform indicates a magnetic flux density of less than 6 milliTesla (mT).

19. The method of claim 16, wherein the magnetic field is operable to stimulate one or more of the follow regions of a brain of the subject: dorsolateral prefrontal cortex, the visual cortex, hippocampus and posterior cortical hot zone.
